(19) United States
(12) Patent Application Publication
(10) Pub. No.: US 2011/0236462 A1
(43) Pub. Date: Sep. 29, 2011

(54) INTRAVAGINAL DRUG DELIVERY DEVICE

(76) Inventors: Ze'ev Shaked, San Antonio, TX (US); Klaus Nickisch, Berlin (DE); James DiNunzio, Cranford, NJ (US); Feng Zhang, Mountain View, CA (US); Marcelo Omelczuk, Austin, TX (US)

(21) Appl. No.: 13/073,899

(22) Filed: Mar. 28, 2011

Related U.S. Application Data
(60) Provisional application No. 61/318,376, filed on Mar. 28, 2010.

(51) Int. Cl. A61F 6/14; A61K 9/00; A61K 31/56

(52) U.S. Cl. 424/432; 424/430; 514/179; 514/182

(57) ABSTRACT

Described herein is an intravaginal drug delivery system. In an embodiment the intravaginal drug delivery system includes a progestin and estrogen compound, and releases the active ingredients in a fixed physiological ratio over a prolonged period of time to produce a contraceptive state in a female.

### PRIORITY CLAIM

This application claims the benefit of U.S. Provisional Application No. 61/318,376 filed on Mar. 28, 2010.

### BACKGROUND OF THE INVENTION

1. Field of the Invention

The present invention generally relates to drug delivery systems. More particularly, the invention relates to vaginal drug delivery systems, which release one or more active substances in a substantially constant ratio over a prolonged period of time.

2. Description of the Relevant Art

Combined oral contraceptive pills, (e.g., oral contraceptives that include a combination of a progestin and an estrogen component) were developed to inhibit normal fertility in women. Such pills inhibit follicular development and prevent ovulation as their primary mechanism of action. Combined oral contraceptive pills are favored over oral contraceptives that include a single dosage (e.g., a gestagen), due to a reduced incidence of breakthrough bleeding and various side effects.

Many of the side effects associated with oral contraceptive pills are due to the use of hormones to regulate the reproductive functions of women. Some of the potential side effects include: depression, vaginal discharge, changes in menstrual flow, breakthrough bleeding, nausea, vomiting, headaches, changes in the breasts, changes in blood pressure, loss of scalp hair, skin problems and skin improvements, increased risk of deep venous thrombosis (DVT) and pulmonary embolism, stroke and myocardial infarction (heart attack). The incidence of various side effects appears to be related, to some extent, on the dosage of both the gestagen and estrogen components. By minimizing the amount of one or both of these compounds administered many of the known side effects may be reduced or eliminated.

In some instances intravaginal delivery provides good adsorption of active agents while avoiding the first-pass effect in the liver. As a result, intravaginal delivery has been considered an efficacious method for administering many types of active agents. Intravaginally administered active agents can directly diffuse through the vaginal tissues to provide a local effect or a systemic effect, thereby treating numerous conditions within and outside the vaginal and/or urogenital tract, such as hormonal dysfunctions, inflammation, infection, pain, and incontinence. Because of the rapid absorption of active agents through the vaginal tissues, and the avoidance of first pass liver and gastric modifications of the active agents, administration of active agents, particularly hormones, through the vaginal tissues may reduce or eliminate some of the side effects associated with oral administration of hormones.

Vaginal delivery systems capable of releasing two or more therapeutically active substances at a substantially constant rate to one another over a prolonged period of time are, for example, useful for certain applications. In particular, such devices would be useful for contraception and hormone replacement therapy. A number of intravaginal delivery systems have been proposed but all tend to suffer from being relatively complicated, making them more expensive to manufacture.

There is a need in the art for improved intravaginal devices capable of delivering active agents to the uterus or vaginal space, with the devices having increased physical integrity, safety, and comfort.

### SUMMARY OF THE INVENTION

In one embodiment, an intravaginal drug delivery device comprises an uncoated thermoplastic matrix; and a progestin dispersed in the thermoplastic matrix. In one embodiment, the progestin compound is etonogestrel. In another embodiment, the progestin compound is levonorgestrel. In one embodiment, the device has a substantially annular form. The device may deliver an effective amount of the progestin for at least 30 days.

In some embodiments, the thermoplastic matrix further comprises an estrogen compound dispersed in the thermoplastic matrix. In one embodiment, the estrogen compound is ethinylestradiol. In an embodiment, the estrogen compound is a nitrated estrogen derivative.

In some embodiments, the thermoplastic matrix comprises an ethylene vinyl acetate copolymer. The thermoplastic matrix may also be composed of one or more hydrophilic matrix materials and/or one or more hydrophobic matrix materials. In an embodiment, the thermoplastic matrix comprises an ethyl vinyl acetate copolymer and one or more hydrophilic matrix materials.

In some embodiments, the thermoplastic matrix includes one or more functional excipients. Examples of functional excipients include pore forming components and biodegradable polymer. Additional active agents may be present in the thermoplastic matrix including, but not limited to, antifungal compounds, and antiprogestins.

In an embodiment, a method of making an intravaginal drug delivery device includes forming a mixture of a thermoplastic polymer and a progestin; heating the thermoplastic polymer/progestin mixture such that at least a portion of the thermoplastic polymer is softened or melted to form a heated mixture of thermoplastic polymer and progestin; and permitting the heated mixture to solidify as a solid mass. In one embodiment, the heated mixture is placed in a mold to form the solid mass.

In one embodiment, the method further includes blending an estrogen compound with the progestin and the thermoplastic polymer. The estrogen compound, in one embodiment, is ethinylestradiol. In another embodiment, the estrogen compound is a nitrated estrogen derivative.

In an embodiment, an intravaginal drug delivery device includes a thermoplastic matrix, a progestin dispersed in the thermoplastic matrix; wherein the concentration of progestin dispersed in the thermoplastic matrix is greater than about 6 times the saturation concentration for the progestin in the thermoplastic matrix; and an estrogen dispersed in the thermoplastic matrix.

In another embodiment, an intravaginal drug delivery device comprises a thermoplastic matrix, a progestin dispersed in the thermoplastic matrix; and an estrogen dispersed in the thermoplastic matrix; wherein the thermoplastic matrix has a non-annular geometry that allows controlled release of the progestin and the estrogen over a predetermined number of days. Non-annular geometries include, but are not limited to a strand of geometrically shaped segments linked together or a half torus.

A method of producing a contraceptive state in a subject includes positioning any intravaginal device, as described above, in the vagina or uterus of a female.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts an intravaginal drug delivery device having an annular geometry;

FIG. 2 depicts an intravaginal drug delivery device having a geometry in the form of a strand of geometrically shaped segments linked together;

FIG. 3 depicts an intravaginal drug delivery device having a half-oval geometry;

FIG. 4 depicts an intravaginal drug delivery device having a hollow cylindrical geometry; and

FIG. 5 depicts an intravaginal drug delivery device having a monolithic film geometry.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a progestin” includes one or more progestins.

As used herein, an “intravaginal device” refers to an object that provides for administration or application of an active agent to the vaginal and/or urogenital tract of a subject, including, e.g., the vagina, cervix, or uterus of a female.

In an embodiment, an intravaginal drug delivery device includes an uncoated thermoplastic matrix, a progestin dispersed in the thermoplastic matrix. Optionally, an estrogen may also be dispersed in the thermoplastic matrix.

A variety of materials may be used as the thermoplastic matrix. Generally, the materials used in the intravaginal device of are suitable for extended placement in the vaginal tract or the uterus. In an embodiment, a thermoplastic material used to form the intravaginal drug delivery device is nontoxic and non-absorbable in the subject. In other embodiments, the intravaginal drug delivery device may be formed from a biodegradable material. In some embodiments, the materials may be suitably shaped and have a flexibility allowing for intravaginal administration.

Suitable materials for use in the formation of an intravaginal drug delivery device include, but are not limited to: polysiloxanes (e.g., poly(dimethyl siloxane); copolymers of dimethylsiloxanes and methylvinylsiloxanes; ethylene/vinyl acetate copolymers (EVA); polyethylene; polypropylene; ethylene/propylene copolymers; acrylic acid polymers; ethylene/ethyl acrylate copolymers; polytetrafluoroethylene (PTFE); polyurethanes; polyesters; polybutadiene; polyisoprene; poly(methacrylate); polymethyl methacrylate; styrene-butadiene-styrene block copolymers; poly(hydroxyethylmethacrylate) (pHEMA); polyvinyl chloride; polyvinyl acetate; polyethers; polyacrylonitriles; polyethylene glycols; polymethylpentene; polybutadiene; polyhydroxy alkanoates; poly(lactic acid); poly(glycolic acid); polyanhydrides; polyorthoesters; hydrophilic hydrogels; cross-linked polyvinyl alcohol; neoprene rubber; butyl rubber; or mixtures thereof.

In an embodiment, an intravaginal drug delivery device is formed from an ethylene/vinyl acetate copolymer (EVA). A variety of grades may be used including grades having a low melt index, a high melt index, a low vinyl acetate content or a high vinyl acetate content. As used herein, EVA having a “low melt index” has a melt index of less than about 100 g/10 min as measured using ASTM test 1238. EVA having a “high melt index” has a melt index of greater than about 100 g/10 min as measured using ASTM test 1238. EVA having a “low vinyl acetate content” has a vinyl acetate content of less than about 20% by weight. EVA having a “high vinyl acetate content” has a vinyl acetate content of greater than about 20% by weight. The thermoplastic matrix of an intravaginal drug delivery device may be formed from EVA having a low melt index, a high melt index, a low vinyl acetate content or a high vinyl acetate content. In some embodiments, the thermoplastic matrix may include: mixtures of a low melt index and high melt index EVA or mixtures of low vinyl acetate content and high vinyl acetate content EVA.

In an embodiment, a combination of one or more suitable materials may be used to form the thermoplastic matrix. The material(s) may be selected to allow prolonged release of the active ingredients from the thermoplastic matrix without the need for an outer controlled release coating. In addition, the concentration of the active agents, in combination with the matrix material may be selected to provide the desired effect.

In one embodiment, the thermoplastic matrix may be composed of ethyl vinyl acetate copolymer in combination with a hydrophobic polymer. For purposes of the present disclosure a matrix material is considered to be hydrophobic or water-insoluble if it is “sparingly soluble” or “practically insoluble” or “insoluble” as defined by USP 29/NF 24.

Examples of hydrophobic polymers include, but are not limited to acrylic acid-based polymers, methacrylic acid based polymers, and acrylic acid-methacrylic acid based copolymers. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid. Derivatives of acrylic acid and methacrylic acid include, but are not limited to, alkyl ester derivatives, alkylether ester derivatives, amide derivatives, alkyl amine derivatives, anhydride derivatives, cyanoalkyl derivatives, and amino-acid derivatives. Examples of acrylic acid-based polymers, methacrylic acid based polymers, and acrylic acid-methacrylic acid based copolymers include, but are nor limited to to Eudragit® L100, Eudragit® L100-55, Eudragit® L 30 D-55, Eudragit® S100, Eudragit® 4135F, Eudragit® RS, acrylic acid and methacrylic acid copolymers, methyl methacrylate polymers, methyl methacrylate copolymers, polyethoxyethyl methacrylate, polycyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polyalkylmethacrylate, polyacrylamide, and polymethacrylic acid anhydride and glycidyl methacrylate copolymers.

Further examples of hydrophobic polymers include, but are not limited to, alkylcelluloses such as ethylcellulose, calcium carboxymethyl cellulose, certain substituted cellulose polymers such as hydroxypropyl methylcellulose phthalate, and hydroxypropyl methylcellulose acetate succinate, cellulose acetate butyrate, cellulose acetate phthalate, and cellulose acetate trimaleate, polyvinyl acetate phthalate, polyvinyl acetate, polyester, shellac, zein, or the like.

In one embodiment, the thermoplastic matrix may be composed of ethyl vinyl acetate copolymer in combination with a hydrophilic polymer. For purposes of the present disclosure, a matrix material is considered hydrophilic and a polymer is considered to be water-soluble if it is more than sparingly soluble as defined by USP 29/NF 24, that is if according to USP 29/NF 24 the matrix material or polymer is classified as “soluble” or “very soluble.” When used in the thermoplastic matrix material the hydrophilic polymer preferably is from about 1% to about 50% of the thermoplastic matrix material by weight, more preferably less than about 30%, less than about 20%; or less than about 10% of the thermoplastic matrix by weight.

Examples of hydrophilic polymers include, but are not limited to polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose (HEC), hydroxymethyl cellulose and hydroxypropyl methylcellulose (HPMC), carboxymethyl cellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

In some embodiments, the thermoplastic matrix may include one or more biodegradable polymers. Examples of biodegradable polymers include, but are not limited to, polylactic acid (PLA), polyglycolic acid (PGA), polyglycolic lactic acid (PGLA), and polycaprolactone.

In an embodiment, the active agents, for example the progestin and, optionally, the estrogen are dispersed in the thermoplastic matrix. As used herein the term “dispersed”, with respect to a polymer matrix, means that a compound is substantially evenly distributed through the polymer, either as a solid suspension in the polymer or dissolved within the polymer matrix. The term “particle dispersion,” as used herein refers to a suspension of the compound particles homogenously distributed in the polymer. The term “molecular dispersion,” as used herein refers to the dissolution of the compound in the polymer. For purposes of this disclosure, a dispersion may be characterized as a particle dispersion if particles of the compound are visible in the polymer at a magnification of about 100× under regular and polarized light. A molecular dispersion is characterized as a dispersion in which substantially no particles of the compound are visible in the polymer at a magnification of 100× under regular and polarized light.

In addition to the thermoplastic matrix and one or more therapeutic agents, one or more functional excipients may be incorporated into the thermoplastic matrix. Examples of excipients include, but are not limited to antioxidants, buffering agents, alkalinizing agents, disintegrants, chelating agents, colorants, surfactants, solubilizers, wetting agents, stabilizers, waxes, lipophilic materials, absorption enhancers, preservatives, absorbents, cross-linking agents, bioadhesive polymers, retardants, pore formers, osmotic agents and fragrance

In one embodiment, one or more pore forming components may be dispersed in the thermoplastic matrix. Exemplary pore forming components include binders such as lactose, calcium sulfate, calcium phosphate and the like; salts such as sodium chloride, magnesium chloride and the like, poloxamers and combinations thereof and other similar or equivalent materials which are widely known in the art.

In an embodiment, the intravaginal drug delivery device is used to produce a contraceptive state in a female mammal. The contraceptive state may be produced by administering an intravaginal drug delivery device that includes a progestin. In other embodiments, contraceptive state may be produced by administering an intravaginal drug delivery device that includes a progestin and an estrogen component.

As used herein, a “progestin” refers to a progestogen, a progestational substance, or any pharmaceutically acceptable substance in the steroid art that generally possesses progestational activity including synthetic steroids that have progestational activity. Progestins suitable for use may be of natural or synthetic origin. Progestins include, but are not limited to: 17α-17-hydroxy-11-methylene-19-norpregna-4,15-dien-20-yn-3-one, 17α-ethynyl-19-nortestosterone, 17α-ethynyltestosterone, 17-deacetylnorgestimate, 19-nor-17-hydroxyprogesterone, 19-norprogesterone, 3β-hydroxydesogestrel, 3-ketodesogestrel (etonogestrel), acetoxypregnenolone, algestone acetophenide, allylestrenol, amgestone, anagestone acetate, chlormadinone, chlormadinone acetate, cyproterone, cyproterone acetate, d-17β-acetoxy-13β-ethyl-17α-ethynylgon-4-en-3-one oxime, demegestone, desogestrel, dienogest, dihydrogesterone, dimethisterone, drospirenone, dydrogesterone, ethisterone (pregneninolone, 17α-ethynyltestosterone), ethynodiol diacetate, fluorogestone acetate, gastrinone, gestadene, gestodene, gestonorone, gestrinone, hydroxymethylprogesterone, hydroxymethylprogesterone acetate, hydroxyprogesterone, hydroxyprogesterone acetate, hydroxyprogesterone caproate, levonorgestrel (l-norgestrol), lynestrenol (lynoestrenol), mecirogestone, medrogestone, medroxyprogesterone, medroxyprogesterone acetate, megestrol, megestrol acetate, melengestrol, melengestrol acetate, nestorone, nomegestrol, norelgestromin, norethindrone (norethisterone) (19-nor-17α-ethynyltestosterone), norethindrone acetate (norethisterone acetate), norethynodrel, norgestimate, norgestrel (d-norgestrel and dl-norgestrel), norgestrienone, normethisterone, progesterone, promegestone, quingestanol, tibolone, and trimegestone. In some embodiments, the progestin is progesterone, etonogestrel, levonorgestrel, gestodene, norethisterone, drospirenone, or combinations thereof.

As used herein, an “estrogen” refers to any of various natural or synthetic compounds that stimulate the development of female secondary sex characteristics and promote the growth and maintenance of the female reproductive system, or any other compound that mimics the physiological effect of natural estrogens. Estrogens also include compounds that can be converted to active estrogenic compounds in the uterine environment. Estrogens include, but are not limited to, estradiol (17β-estradiol), estridiol acetate, estradiol benzoate, estridiol cypionate, estridiol decanoate, estradiol diacetate, estradiol heptanoate, estradiol valerate, 17α-estradiol, estriol, estriol succinate, estrone, estrone acetate, estrone sulfate, estropipate (piperazine estrone sulfate), ethynylestradiol (17α-ethynylestradiol, ethinylestradiol, ethinyl estradiol, ethynyl estradiol), ethynylestradiol 3-acetate, ethynylestradiol 3-benzoate, mestranol, quinestrol, and nitrated estrogen derivatives.

Nitrated estrogen derivatives are described in U.S. Pat. No. 5,554,603 to Kim et al. which is incorporated herein by reference. Nitrated estrogen derivatives that may be used in combination with a progestin include compounds having the structure:

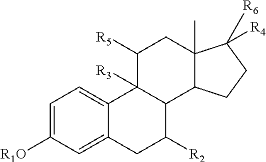

where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

In some embodiments, the nitrated estrogen derivative has the structure:

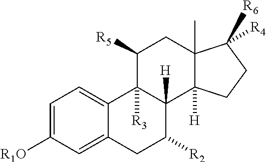

where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

A specific compound that may be used in combination with a progestin in an oral contraceptive to inhibit ovulation in a female subject includes the compound (+)-3,11β,17β-trihydroxyestra-1,3,5(10)-triene 3-acetate-11,17-dinitrate ester, which has the structure:

Other active agents may be incorporated into the intravaginal drug delivery device including antiprogestins, antibiotics, and antifungal compounds. As used herein “antiprogestins” are compounds that act as progesterone antagonists. Such compounds may be particular useful as contraceptives as well as for the treatment of various types of cancers. If incorporated into an intravaginal drug delivery device, such compounds may help treat cancers such as cervical cancer or breast cancers. Examples, of antiprogestins include, but are not limited to, Mifepristone, Onapristone, ORG-33628, Proellex, and Lonaprisan (ZK-230211).

Other antiprogestins that may be incorporated into the intravaginal drug delivery device include antiprogestins that are described in U.S. Patent Application Publication No. 2010/0273759 entitled “Progesterone Antagonists”, which is incorporated herein by reference. Exemplary progesterone antagonists that may be incorporated into the intravaginal drug delivery device include compounds having the structure:

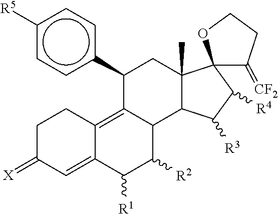

In which
R¹ is a hydrogen atom, a straight-chain C₁-C₅ alkyl group, a branched C₁-C₅ alkyl group, a C₃-C₅ cycloalkyl group, or a halogen atom;
R² is a hydrogen atom, a straight-chain C₁-C₅ alkyl group a branched C₁-C₅ alkyl group, a C₃-C₅ cycloalkyl group, or a halogen atom; or
R¹ and R² together are a methylene group;
R³ is a hydrogen atom, a straight-chain C₁-C₅ alkyl group a branched C₁-C₅ alkyl group, a C₃-C₅ cycloalkyl group, or a halogen atom;
R⁴ is a hydrogen atom, a straight-chain C₁-C₅ alkyl group a branched C₁-C₅ alkyl group, a C₃-C₅ cycloalkyl group, or a halogen atom; or
R³ and R⁴ together are an additional bond or a methylene group;
R⁵ is a radical Y or an aryl radical that is optionally substituted with Y, wherein Y is a hydrogen atom, a halogen atom, —OR⁶, —NO₂, —N₃, —CN, —NR^{6a}R^{6b}, —NHSO₂R⁶, —CO₂R⁶, C₁-C₁₀ alkyl, C₁-C₁₀ substituted alkyl, C₁-C₁₀ cycloalkyl, C₁-C₁₀ alkenyl, C₁-C₁₀ alkynyl, C₁-C₁₀ alkoxy, C₁-C₁₀ alkanoyloxy, benzoyloxy, arylacyl, C₁-C₁₀-alkylacyl, C₁-C₁₀-cycloalkylacyl, C₁-C₁₀ hydroxyalkyl, aryl or arylalkyl, a five or six membered heterocyclic radical containing up to three heteroatoms;
R^{6a} and R^{6b} are the same or different and represent a hydrogen atom or a C₁-C₁₀ alkyl group, R⁶ is a hydrogen atom or C₁-C₁₀ alkyl,
when Y is a —NR^{6a}R^{6b} radical, Y may be in the form of a physiologically compatible salt formed by reaction of an acid;
when Y is —CO₂R⁶, R⁶ may represent a cation of a physiologically compatible salts formed by reaction with a base; and

the wavy lines represent that the substituent can be in an α- or β-orientation.

Examples of antifungal compounds include, but are not limited to polyene antifungals such as natamycin, rimocidin, filipin, nystatin, amphotericin B, candicin, and hamycin; imidazole antifungals such as miconazole (Micatin®), ketoconazole (Nizoral®, Fungoral® and Sebizole®), clotrimazole (Lotrimin®, Lotrimin AF® and Canesten®), econazole, omoconazole, bifonazole, butoconazole, fenticonazole, isoconazole, oxiconazole, sertaconazole (Ertaczo®), sulconazole, and tioconazole; triazole antifungals such as fluconazole, itraconazole, isavuconazole, ravuconazole, posaconazole, voriconazole, terconazole, and albaconazole; thiazole antifungals such as abafungin; allylamine antifungals such as terbinafine (Lamisil®), naftifine (Naftin®), and butenafine (Lotrimin Ultra®); and echinocandin antifungals such as anidulafungin, caspofungin, and micafungin. Other compounds that have antifungal properties include, but are not limited to polygodial, benzoic acid, ciclopirox, tolnaftate (Tinactin®, Desenex® and Aftate®), undecylenic acid, flucytosine or 5-fluorocytosine, griseofulvin, and haloprogin.

Examples of antibiotic compounds include but are not limited to β-lactam antibiotics such as benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (amoxicillin+clavulanic acid), azlocillin, carbenicillin, ticarcillin, mezlocillin, piperacillin, cephalosporin, cephalexin, cephalothin, cefazolin, cefaclor, cefuroxime, cefamandole, cefotetan, cefoxitin, ceftriaxone, cefotaxime, cefpodoxime, cefixime, ceftazidime, cefepime, cefpirome, carbapenem, imipenem (with cilastatin), meropenem, ertapenem, faropenem, doripenem, aztreonam (Azactam®), tigemonam, nocardicin A, tabtoxinine-β-lactam, clavulanic acid, tazobactam, and sulbactam; Aminoglycoside antibiotics such as aminoglycoside, amikacin, apramycin, arbekacin, astromicin, bekanamycin, capreomycin, dibekacin, dihydrostreptomycin, elsamitrucin, G418, gentamicin, hygromycin B, isepamicin, kanamycin, kasugamycin, micronomicin, neomycin, netilmicin, paromomycin sulfate, ribostamycin, sisomicin, streptoduocin, streptomycin, tobramycin, verdamicin; sulfonamides such as sulfamethoxazole, sulfisomidine (also known as sulfaisodimidine), sulfacetamide, sulfadoxine, dichlorphenamide (DCP), and dorzolamide; quinolone antibiotics such as cinobac, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, garenoxacin, and delafloxacin; and oxazolidone antibiotics such as linezolid, torezolid, eperezolid, posizolid, and radezolid.

The intravaginal delivery device can be in any shape suitable for insertion and retention in the vaginal tract without causing undue discomfort to the user. For example, the intravaginal device may be flexible. As used herein, “flexible” refers to the ability of an intravaginal drug delivery device to bend or withstand stress and strain without being damaged or broken. For example, an intravaginal may be deformed or flexed, such as, for example, using finger pressure, and upon removal of the pressure, return to its original shape. The flexible properties of the intravaginal drug delivery device are useful for enhancing user comfort, and also for ease of administration to the vaginal tract and/or removal of the device from the vaginal tract.

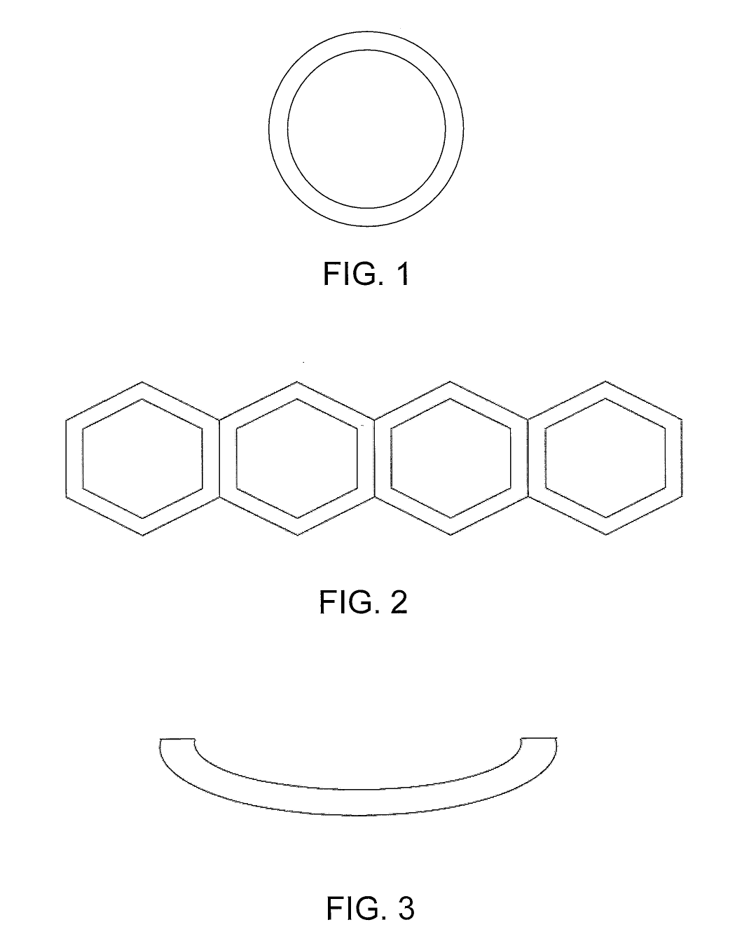

In an embodiment, the intravaginal drug delivery device may be annular in shape. As used herein, “annular” refers to a shape of, relating to, or forming a ring. Annular shapes suitable for use include a ring, an oval, an ellipse, a toroid, and the like. In some embodiments, the intravaginal drug delivery device is a vaginal ring, as depicted in FIG. 1.

The intravaginal drug delivery device may have a non-annular geometry. Examples of non-annular geometries are depicted in FIGS. 2-4. In one embodiment, the thermoplastic matrix used to form the intravaginal drug delivery device has a geometry in the form of a strand of geometrically shaped segments linked together. For example, as shown in FIG. 1 a plurality of hexagon shaped units may be linked to form a strand. Other geometrically shaped units including, but not limited to, squares, triangles, rectangles, pentagons, heptagons, octagons, etc. may be formed into strands. In some embodiment, mixtures of different geometrically shaped units may be joined to together in a strand. The strand of geometrically shaped units may be joined together to form ring-like structure.

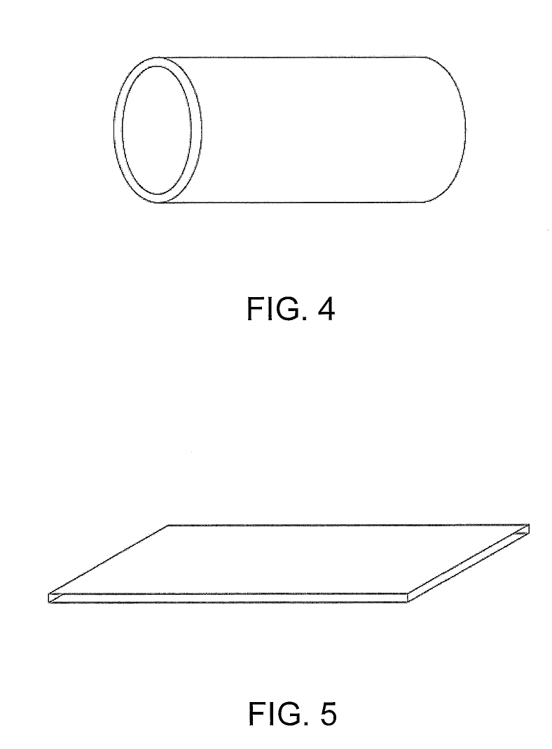

FIG. 3 depicts another embodiment of an intravaginal drug delivery device in the shape of a half oval. A half oval device may be easier to manufacture than a full ring. In an embodiment, the half oval shape may allow a user to form a ring like structure before and/or after insertion. FIG. 4 depicts another embodiment of an intravaginal drug delivery device in the shape of a hollow cylinder. Use of a hollow cylinder may allow easier insertion of the intravaginal delivery device. The hollow cylinder geometry may allow insertion of the intravaginal drug delivery device into the vaginal tract in a compressed form, which, upon deployment, expands inside the tract to improve the retention of the device. FIG. 5 depicts a monolithic film geometry. Such a film may be formed or include, mucoadhesive substances to improve adhesion to the vaginal tract.

The intravaginal drug delivery device may be manufactured by any known techniques. In some embodiments, therapeutically active agent(s) may be mixed within the thermoplastic matrix material and processed to the desired shape by: injection molding, rotation/injection molding, casting, extrusion, or other appropriate methods. In one embodiment, the intravaginal drug delivery device is produced by a hot-melt extrusion process.

In one embodiment, a method of making an intravaginal drug delivery device includes:
a. forming a mixture of a thermoplastic polymer and a progestin;
b. heating the thermoplastic polymer/progestin mixture such that at least a portion of the thermoplastic polymer is softened or melted to form a heated mixture of thermoplastic polymer and progestin; and;
c. permitting the heated mixture to cool and solidify as a solid mass,
d. and optionally, shaping the mass into a predetermined geometry.

For purposes of the present disclosure a mixture is “softened” or “melted” by applying thermal or mechanical energy sufficient to render the mixture partially or substantially completely molten. For instance, in a mixture that includes a matrix material, “melting” the mixture may include substantially melting the matrix material without substantially melting one or more other materials present in the mixture (e.g., the therapeutic agent and one or more excipients). For polymers, a “softened” or “melted” polymer is a polymer that is heated to a temperature at or above the glass transition temperature of the polymer. Generally, a mixture is sufficiently melted or softened, when it can be extruded as a continuous rod, or when it can be subjected to injection molding.

The mixture of the thermoplastic polymer and the progestin can be produced using any suitable means. Well-known mixing means known to those skilled in the art include dry mixing, dry granulation, wet granulation, melt granualation, high shear mixing, and low shear mixing.

Granulation generally is the process wherein particles of powder are made to adhere to one another to form granules, typically in the size range of 0.2 to 4.0 mm. Granulation is desirable in pharmaceutical formulations because it produces relatively homogeneous mixing of different sized particles.

Dry granulation involves aggregating powders with high compressional loads. Wet granulation involves forming granules using a granulating fluid including either water, a solvent such as alcohol or water/solvent blend, where this solvent agent is subsequently removed by drying. Melt granulation is a process in which powders are transformed into solid aggregates or agglomerates while being heated. It is similar to wet granulation except that a binder acts as a wetting agent only after it has melted. The granulation is further achieved following using milling and/or screening to obtain the desired particle sizes or ranges. All of these and other methods of mixing pharmaceutical formulations are well-known in the art.

Subsequent or simultaneous with mixing, the mixture of thermoplastic polymer and the progestin is softened or melted to produce a mass sufficiently fluid to permit shaping of the mixture and/or to produce melding of the components of the mixture. The softened or melted mixture is then permitted to solidify as a substantially solid mass. The mixture can optionally be shaped or cut into suitable sizes during the softening or melting step or during the solidifying step. In some embodiments, the mixture becomes a homogeneous mixture either prior to or during the softening or melting step. Methods of melting and molding the mixture include, but are not limited to, hot-melt extrusion, injection molding and compression molding.

Hot-melt extrusion typically involves the use of an extruder device. Such devices are well-known in the art. Such systems include mechanisms for heating the mixture to an appropriate temperature and forcing the melted feed material under pressure through a die to produce a rod, sheet or other desired shape of constant cross-section. Subsequent to or simultaneous with being forced through the die the extrudate can be cut into smaller sizes appropriate for use as an oral dosage form. Any suitable cutting device known to those skilled in the art can be used, and the mixture can be cut into appropriate sizes either while still at least somewhat soft or after the extrudate has solidified. The extrudate may be cut, ground or otherwise shaped to a shape and size appropriate to the desired oral dosage form prior to solidification, or may be cut, ground or otherwise shaped after solidification. In some embodiments, an oral dosage form may be made as a non-compressed hot-melt extrudate. In other embodiments, an oral dosage form is not in the form of a compressed tablet.

Injection molding typically involves the use of an injection-molding device. Such devices are well-known in the art. Injection molding systems force a melted mixture into a mold of an appropriate size and shape. The mixture solidifies as least partially within the mold and then is released.

Compression molding typically involves the use of an compression-molding device. Such devices are well-known in the art. Compression molding is a method in which the mixture is optionally preheated and then placed into a heated mold cavity. The mold is closed and pressure is applied. Heat and pressure are typically applied until the molding material is cured. The molded oral dosage form is then released from the mold.

The final step in the process of making intravaginal drug delivery device is permitting the mixture to solidify as a solid mass. The mixture may optionally be shaped either prior to solidification or after solidification. Solidification will generally occur either as a result of cooling of the melted mixture or as a result of curing of the mixture however any suitable method for producing a solid dosage form may be used.

In preferred embodiments, the intravaginal drug delivery device includes a progestin as a substantially uniform dispersion within the thermoplastic matrix. However in alternative embodiments the distribution of the progestin within the thermoplastic matrix can be substantially non-uniform. One method of producing a non-uniform distribution of the progestin is through the use of one or more coatings of water-insoluble or water-soluble polymer. Another method is by providing two or more mixtures of polymer or polymer and progestin to different zones of a compression or injection mold. These methods are provided by way of example and are not exclusive. Other methods of producing a non-uniform distribution of therapeutic agent within the abuse-deterring oral dosage forms will be apparent to those skilled in the art.

In practice, for a human female, an annular intravaginal drug delivery device has an outer ring diameter from 35 mm to 70 mm, from 35 mm to 60 mm, from 45 mm to 65 mm, or from 50 mm to 60 mm. The cross sectional diameter may be from 1 mm to 10 mm, from 2 mm to 6 mm, from 3.0 mm to 5.5 mm, from 3.5 mm to 4.5 mm, or from 4.0 mm to 5.0 mm.

The amount of active agent released from the intravaginal drug delivery device may be determined by a qualified healthcare professional and is dependent on many factors, e.g., the active agent, the condition to be treated, the age and/or weight of the subject to be treated, etc. In some embodiments, the active agent is released from the device at an average rate of about 0.01 mg to about 10 mg per 24 hours in situ, or about 0.05 mg to about 5 mg per 24 hours in situ, or about 0.1 mg to about 1 mg per 24 hours in situ. In some embodiments, the active agent is released from the device at an average rate of about 1 mg to about 100 mg per 24 hours in situ or about 5 mg to about 50 mg per 24 hours in situ.

In some embodiments, two or more active agents can be released from the device at a different rate per 24 hours in situ. For example, an estrogen can be released from the device at an average rate of about 0.01 mg to about 0.1 mg per 24 hours and a progestin can be released from the device at an average rate of about 0.08 mg to about 0.2 mg per 24 hours in situ, or an estrogen can be released from the device at an average rate of about 0.1 mg to about 1 mg per 24 hours in situ and a progestin can be released from the device at an average rate of about 0.05 mg to about mg per 24 hours in situ, or an estrogen can be released from the device at an average rate of about 0.05 mg to about 5 mg per 24 hours in situ and a progestin can be released from the device at an average rate of about 1 mg to about 100 mg per 24 hours in situ.

The release rate can be measured in vitro using, e.g., the USP Apparatus Paddle 2 method. The active agent(s) can be assayed by methods known in the art, e.g., by HPLC.

In some embodiments of the present invention, active agent(s) is/are released from the intravaginal device at a steady rate for up to about 1 month or about 30 days after administration to a female, for up to about 25 days after administration to a female, for up to about 21 days after administration to a female, for up to about 15 days after administration to a female, for up to about 10 days after administration to a female, for up to about 7 days after administration to a female, or for up to about 4 days after administration to a female.

As used herein, a “steady rate” is a release rate that does not vary by an amount greater than 70% of the amount of active agent released per 24 hours in situ, by an amount greater than 60% of the amount of active agent released per 24 hours in situ, by an amount greater than 50% of the amount of active agent released per 24 hours in situ, by an amount greater than 40% of the amount of active agent released per 24 hours in situ, by an amount greater than 30% of the amount of active agent released per 24 hours in situ, by an amount greater than 20% of the amount of active agent released per 24 hours in situ, by an amount greater than 10% of the amount of active agent released per 24 hours in situ, or by an amount greater than 5% of the amount of active agent released per 24 hours in situ

In some embodiments, the active agent is a progestin with a steady release rate of active agent in situ of about 80 μg to about 200 μg per 24 hours, about 90 μg to about 150 μg per 24 hours, about 90 μg to about 125 μg per 24 hours, or about 95 μg to about 120 μg per 24 hours.

In some embodiments, the active agent includes an estrogen with a steady release rate of active agent in situ of about 10 μg to about 100 μg per 24 hours, about 10 μg to about 80 μg per 24 hours, about 10 μg to about 60 μg per 24 hours, about 10 μg to about 40 μg per 24 hours, about 10 μg to about 20 μg per 24 hours, or about 10 μg to about 15 μg per 24 hours.

Use of an intravaginal drug delivery device that includes progestin without an estrogen has advantages over combined progestin/estrogen devices. Some women are unable to tolerate estrogen. For example, women that are breast feeding are unable to take contraceptives that include estrogen. For such women, use of an intravaginal drug delivery device that includes only a progestin would offer a safe solution to the desire to have effective birth control while being unable to take estrogen containing formulations.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

A progestin and an estrogen are embedded into an ethylene vinyl acetate (EVA) matrix using a melt extruder, using the levels provided in formulation Table 1 below:

TABLE 1

| Material | % | Amount |
|---|---|---|
| Progestin | 0.175 | 2.520 |
| Estrogen | 0.021875 | 0.315 |
| EVA | 99.803125 | 1,437.165 |
| Total | 100 | 1,440 |

The composition is extruded as a flat monolithic sheet that and provides surface area necessary for sustained release of both drug substances over a period of 21 days when measured by drug release in a volumetric flask in pH 7.4 phosphate buffer.

### Example 2

A progestin was embedded into an ethylene vinyl acetate (EVA) matrix using a melt extruder, using the levels provided in formulation Table 1 below:

TABLE 1

| Material | % | Amount |
|---|---|---|
| Progestin | 0.175 | 2.52 |
| EVA | 99.825 | 1,437.48 |
| Total | 100 | 1,440 |

The composition is extruded and molded into a ring. The resulting device is an uncoated ring of progesterone in an EVA matrix. The ring delivered the progestin over a period of 21 days when measured by drug release in a volumetric flask in pH 7.4 phosphate buffer.

### Example 3

A progestin and an estrogen are embedded into an ethylene vinyl acetate (EVA) matrix using a melt extruder. Additional pore forming agents were incorporated using the levels provided in formulation Table 2 below:

TABLE 2

| Material | % | Amount |
|---|---|---|
| Progestin | 0.175000 | 2.520 |
| Estrogen | 0.021875 | 0.315 |
| Povidone K 29/32 | 10.000000 | 144.000 |
| EVA | 89.803125 | 1,293.165 |
| Total | 100 | 1,440 |

The composition is extruded as a flat monolithic sheet that and provides surface area necessary for sustained release of both drug substances over a period of 21 days when measured by drug release in a volumetric flask in pH 7.4 phosphate buffer.

### Example 4

A progestin and estrogen are embedded into an ethylene vinyl acetate (EVA) matrix using a melt extruder. Additional pore forming agents were incorporated using the levels provided in formulation Table 3 below:

TABLE 3

| Material | % | Amount |
|---|---|---|
| Progestin | 1.500 | 21.6 |
| Estrogen | 0.1875 | 2.7 |
| Povidone K 29/32 | 10.0000 | 144.0 |
| EVA | 88.3125 | 1,271.7 |
| Total | 100 | 1,440.0 |

The composition is extruded as a flat monolithic sheet that and provides surface area necessary for sustained release of both drug substances over a period of 21 days when measured by drug release in a volumetric flask in pH 7.4 phosphate buffer.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An intravaginal drug delivery device comprising:
an uncoated thermoplastic matrix; and
a progestin dispersed in the thermoplastic matrix.

2. The device of claim 1, wherein the progestin compound is etonogestrel.

3. The device of claim 1, wherein the progestin compound is levonorgestrel.

4. The device of claim 1, wherein the thermoplastic matrix further comprises an estrogen compound dispersed in the thermoplastic matrix.

5. The device of claim 4, wherein the estrogen compound is ethinylestradiol.

6. The device of claim 4, wherein the estrogen compound comprises a nitrated estrogen derivative having the structure: 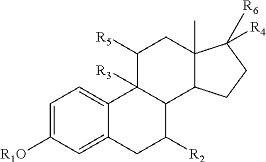
where R₁ is hydrogen, C₁-C₈ alkyl, cycloalkyl, or C₁-C₈ acyl;
R₂ is hydrogen or C₁-C₈ alkyl;
R₃ is hydrogen, hydroxy or C₁-C₈ alkyl;
R₄ is hydrogen or C₁-C₈ alkyl;
where each R₅ and R₆ is, independently, hydrogen or nitrate; and wherein at least one of R₅ and R₆ is a nitrate group.

7. The device of claim 1, wherein the thermoplastic matrix comprises an ethylene vinyl acetate copolymer.

8. The device of claim 1, wherein the thermoplastic matrix comprises one or more hydrophilic matrix materials.

9. The device of claim 1, wherein the thermoplastic matrix comprises one or more hydrophobic matrix materials.

10. The device of claim 1, wherein the thermoplastic matrix comprises an ethyl vinyl acetate copolymer and one or more hydrophilic matrix materials.

11. The device of claim 1, wherein the device has a substantially annular form.

12. The device of claim 1, wherein the thermoplastic matrix further comprises a pore forming component.

13. The device of claim 1, wherein the thermoplastic matrix further comprises a biodegradable polymer.

14. The device of claim 1, wherein the device delivers an effective amount of the progestin for at least 30 days.

15. The device of claim 1, wherein the thermoplastic matrix further comprises one or more antifungal compounds.

16. The device of claim 1, wherein the thermoplastic matrix further comprises one or more antibiotic compounds.

17. The device of claim 1, wherein the thermoplastic matrix further comprises one or more antiprogestin compounds.

18. A method of making an intravaginal drug delivery device comprising:
forming a mixture of a thermoplastic polymer and a progestin;
heating the thermoplastic polymer/progestin mixture such that at least a portion of the thermoplastic polymer is softened or melted to form a heated mixture of thermoplastic polymer and progestin; and
permitting the heated mixture to solidify as a solid mass.

19-33. (canceled)

34. An intravaginal drug delivery device made by the method comprising:
forming a mixture of a thermoplastic polymer and a progestin;
heating the thermoplastic polymer/progestin mixture such that at least a portion of the thermoplastic polymer is softened or melted to form a heated mixture of thermoplastic polymer and progestin; and
permitting the heated mixture to solidify as a solid mass;
wherein the device comprises the progestin dispersed in an uncoated thermoplastic matrix.

35-50. (canceled)

51. A method of producing a contraceptive state in a subject comprising positioning an intravaginal drug delivery device in the vagina or uterus of a female, wherein the intravaginal drug delivery device comprises an uncoated thermoplastic matrix, and a progestin dispersed in the thermoplastic matrix.

52-82. (canceled)
